(19) United States
(12) Patent Application Publication
(10) Pub. No.: US 2020/0138512 A1
(43) Pub. Date: May 7, 2020

(54) Attaining Higher Impedances for Large Indifferent Electrodes

(71) Applicant: Biosense Webster (Israel) Ltd., Yokneam (IL)

(72) Inventors: Christopher Thomas Beeckler, Brea, CA (US); Athanassios Papaioannou, Los Angeles, CA (US)

(21) Appl. No.: 16/182,440

(22) Filed: Nov. 6, 2018

(51) Int. Cl. A61B 18/14; G01N 27/02

(52) U.S. Cl. CPC A61B 18/1492; A61B 2018/00577; G01N 27/02; A61B 18/1402

(57) ABSTRACT

Described embodiments include an apparatus that includes an electrically-conductive layer, including a first face and a second face that are opposite one another, a first electrically-insulative layer that is shaped to define a plurality of apertures and that covers the first face without covering portions of the first face that are aligned with the apertures, and a second electrically-insulative layer that covers the second face. Other embodiments are also described.

### FIELD OF THE INVENTION

The present invention relates to medical procedures, such as ablation procedures, that involve the use of electrodes.

### BACKGROUND

In some medical procedures, such as unipolar cardiac ablation procedures, electric current is passed between a first electrode, which is in contact with internal tissue of a subject, and a second electrode, which is coupled to the surface of the body of the subject. The second electrode may be referred to as a “neutral electrode,” a “return electrode,” or an “indifferent electrode.”

US Patent Application Publication 2014/0342128 describes a microarray structure including a substrate material layer, a continuous three-dimensional (3D) surface layer on the substrate material layer that is capable of functionalization for use as an array, and an inert material, wherein the structure includes functionalizable isolated areas which are between a nanometer and millimeter in size. The functionalizable areas are part of the continuous 3D surface layer and are isolated by the inert material and are interconnected within the structure by the continuous 3D surface layer.

### SUMMARY OF THE EMBODIMENTS

There is provided, in accordance with some embodiments of the present invention, an apparatus that includes an electrically-conductive layer, including a first face and a second face that are opposite one another, a first electrically-insulative layer that is shaped to define a plurality of apertures and that covers the first face without covering portions of the first face that are aligned with the apertures, and a second electrically-insulative layer that covers the second face.

In some embodiments, the electrically-conductive layer includes an electrically-conductive plate,

the first electrically-insulative layer includes a first electrically-insulative cover coupled to the first face of the plate, and the second electrically-insulative layer includes a second electrically-insulative cover coupled to the second face of the plate.

In some embodiments, the plate includes one or more side faces disposed between the first face and the second face, and the second electrically-insulative cover covers the side faces.

In some embodiments, the apparatus includes an electrically-insulative case that includes the first cover and the second cover.

In some embodiments, the second electrically-insulative layer includes an electrically-insulative substrate, the electrically-conductive layer includes an electrically-conductive coating that coats the electrically-insulative substrate, and the first electrically-insulative layer includes an electrically-insulative cover coupled to the electrically-conductive coating.

In some embodiments, the electrically-conductive coating includes a vapor deposition coating.

In some embodiments, the electrically-insulative substrate includes a polyimide.

In some embodiments, the electrically-conductive coating includes copper.

In some embodiments, the first electrically-insulative layer includes an electrically-insulative substrate, the electrically-conductive layer includes an electrically-conductive coating that coats the electrically-insulative substrate, and the second electrically-insulative layer includes an electrically-insulative cover coupled to the electrically-conductive coating.

In some embodiments, the electrically-insulative substrate includes a polyimide.

In some embodiments, the electrically-conductive coating includes copper.

In some embodiments, the electrically-insulative substrate includes a first surface and a second surface that are opposite one another, the electrically-conductive coating coats the first surface of the electrically-insulative substrate, and

the apparatus further includes:
a plurality of electrically-conducting islands that coat respective portions of the second surface of the electrically-insulative substrate that surround the apertures; and
respective metallic deposits that fill the apertures and electrically connect the electrically-conductive coating to the islands.

In some embodiments, the metallic deposits further cover the islands.

In some embodiments, the apparatus further includes respective electrically-conductive metallic deposits that contact the electrically-conductive layer and at least partly fill the apertures.

In some embodiments, the metallic deposits include gold.

In some embodiments, the metallic deposits further cover respective portions of the first electrically-insulative layer that surround the apertures.

In some embodiments, a combined surface area of the portions of the first face that are aligned with the apertures is less than approximately 1% of a total surface area of the first face.

In some embodiments, the combined surface area of the portions of the first face that are aligned with the apertures is less than approximately 0.5% of the total surface area of the first face.

In some embodiments, a distance between any one of the apertures and another, closest one of the apertures is less than approximately 6 mm.

In some embodiments, the total surface area of the first face is at least 9 cm².

In some embodiments, the apertures are arranged in a rectangular grid.

In some embodiments, the apertures are arranged in a hexagonal close-packed pattern.

In some embodiments, the electrically-insulative cover includes a perforated electrically-insulative sheet.

In some embodiments, the electrically-insulative cover includes an electrically-insulative coating.

In some embodiments, the electrically-insulative coating includes a layer of electrically-insulative paint.

There is further provided, in accordance with some embodiments of the present invention, a method for testing an ablation probe. The method includes providing an electrode that includes an electrically-conductive layer, including a first face and a second face that are opposite one another, an electrically-insulative cover that is shaped to define a plurality of apertures and that covers the first face without covering portions of the first face that are aligned with the apertures, and an electrically-insulative layer that covers the second face. The method further includes coupling the electrode and a piece of biological tissue to one another such that the first face faces the piece of biological tissue, placing the electrode and the piece of biological tissue into a bath, and, while the electrode and the piece of biological tissue are coupled to one another in the bath, using the ablation probe, ablating the piece of biological tissue by passing an electric current between the ablation probe and the electrode.

In some embodiments, the first face faces a surface of the piece of biological tissue, and a difference between (i) a total surface area of the first face, and (ii) a surface area of the surface of the piece of biological tissue, is less than approximately 25% of the total surface area of the first face.

There is further provided, in accordance with some embodiments of the present invention, a method that includes providing one or more electrodes, each of the electrodes including an electrically-conductive layer, including a first face and a second face that are opposite one another, an electrically-insulative cover that is shaped to define a plurality of apertures and that covers the first face without covering portions of the first face that are aligned with the apertures, and an electrically-insulative layer that covers the second face. The method further includes coupling each of the electrodes to a body of a subject such that the first face faces the subject and, while the electrodes are coupled to the body of the subject, using an ablation probe disposed within the body, ablating tissue of the subject by passing an electric current between the ablation probe and the electrodes.

In some embodiments, coupling each of the electrodes to the body of the subject includes coupling a first one of the electrodes to a chest of the subject and a second one of the electrodes to a back of the subject.

In some embodiments, coupling each of the electrodes to the body of the subject includes coupling a first one of the electrodes to a forehead of the subject and a second one of the electrodes to a nape of a neck of the subject.

In some embodiments, the tissue is of a type selected from the group of tissue types consisting of: cardiac tissue, otolaryngological tissue, and neurological tissue.

The present invention will be more fully understood from the following detailed description of embodiments thereof, taken together with the drawings, in which:

### BRIEF DESCRIPTION OF THE DRAWINGS

FIG. 1 is a schematic illustration of a method for testing an ablation probe, in accordance with some embodiments of the present invention;

FIGS. 2A-B are schematic illustrations of cross-sections through indifferent electrodes, in accordance with some embodiments of the present invention;

FIGS. 3-4 are schematic exploded views of indifferent electrodes, in accordance with some embodiments of the present invention;

FIG. 5 is a schematic illustration of a cross-section through an indifferent electrode, in accordance with some embodiments of the present invention; and

FIG. 6 is a schematic illustration of an ablation procedure, in accordance with some embodiments of the present invention.

### DETAILED DESCRIPTION OF EMBODIMENTS

### Glossary

As used herein, each of the terms “about” and “approximately,” when applied to any numerical value or range of values used to describe the properties of a component or collection of components, indicates a suitable dimensional tolerance that allows the component or collection of components to function for its intended purpose as described herein. More specifically, “about” or “approximately” may refer to a range of values ranging over ±10% of the stated value, such that, for example, “about 90%” may refer to the range of values from 81% to 99%.

Although the in vivo procedures described herein are typically performed on human subjects, it is noted that the scope of the present disclosure also includes performing these procedures on animal subjects. Thus, it should be understood that, as used herein, each of the terms “patient,” “host,” “user,” and “subject” may refer to any human or animal subject.

### Overview

Typically, when performing in vitro testing of an ablation probe, a piece of biological tissue (comprising, for example, a bovine or porcine heart), together with an indifferent electrode (comprising, for example, a metal plate), are placed in a bath of saline and/or blood. Subsequently, an ablation electrode at the distal end of the probe, which is connected to an ablation-current generator, is brought into contact with the biological tissue. The biological tissue is then ablated, by passing electric currents, which are generated by the generator, between the ablation electrode and the indifferent electrode.

Typically, it is desired that the impedance seen by the generator be generally constant over the surface of the biological tissue. In other words, it is desired that the impedance should not vary significantly as a function of the location on the biological tissue at which the ablation electrode is located. Consequently, the biological tissue and the indifferent electrode are made to have approximately the same size, and the indifferent electrode is made to contact the biological tissue. However, although this setup helps achieve a uniform impedance, a disadvantage of this configuration is that the impedance may be significantly lower than a normal physiological impedance, such that it may be difficult to accurately simulate an in vivo setting. For example, the impedance for the above-described setup may be between 20 and 80 n, whereas a normal physiological impedance for a human subject is between 50 and 150Ω. Hypothetically, the saline and/or blood in the bath could be diluted (e.g., with deionized water) to raise the impedance, but this hypothetical setup would also fail to accurately simulate an in vivo setting.

To address this challenge, embodiments described herein provide an indifferent electrode that provides a uniform yet sufficiently high impedance, such as a uniform impedance that is between 50 and 150Ω, for the above-described in vitro testing. In some embodiments, the electrode comprises an electrically-conducting plate having one face that is covered by an electrically-insulative cover shaped to define a large number of uniformly-distributed small apertures, and another face that is completely covered by an unperforated electrically-insulative cover. Prior to performing the in vitro testing, the electrode is coupled to the biological tissue such that the cover having the apertures contacts the biological tissue. Thus, on the one hand, since the apertures are uniformly distributed, the impedance seen by the generator is uniform, while on the other hand, since the apertures expose only a very small portion of the plate, the impedance is similar to a normal physiological impedance.

Several alternate embodiments, which do not necessarily comprise an electrically-conductive plate, are also described below. For example, in some embodiments, the indifferent electrode comprises an electrically-insulative substrate comprising a surface that is coated by an electrically-conductive coating, which is in turn covered by a perforated cover. In these embodiments, the electrically-conductive coating serves the role of the aforementioned plate, while the substrate serves the role of the unperforated electrically-insulative cover.

In addition to facilitating in vitro testing, the indifferent electrode described herein may be used during an actual ablation procedure. One advantage of using such an electrode is that the apertures spatially distribute the current that passes through the skin of the patient, such as to reduce the chances of any burning. Another advantage is that multiple such electrodes may be spatially distributed over the body of the patient—thus attaining a more uniform impedance—without overly decreasing the impedance that is seen by the generator.

### The Indifferent Electrode

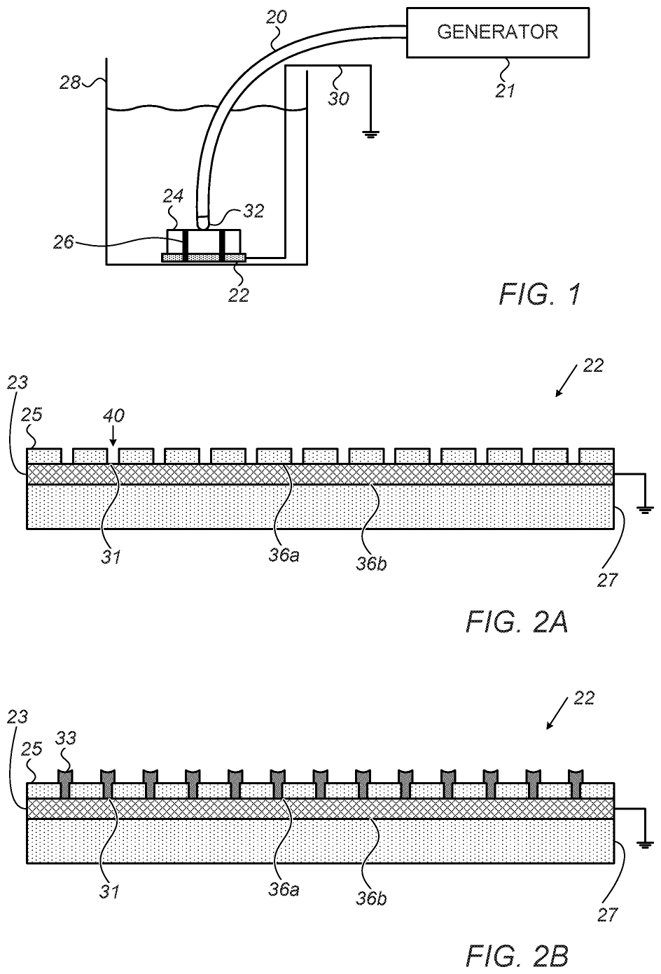

Reference is initially made to FIG. 1, which is a schematic illustration of a method for testing an ablation probe 20, in accordance with some embodiments of the present invention.

Per the method depicted in FIG. 1, probe 20 is connected to a signal generator 21, and an electrode 22—which may also be referred to as an “electrode patch”—is connected, via a wire 30, to electrical ground, such that electrode 22 functions as an indifferent electrode. Electrode 22 and a piece of biological tissue 24 are coupled to one another, e.g., using one or more straps 26. Subsequently to, or prior to, coupling electrode 22 and biological tissue 24 to one another, the electrode and the piece of biological tissue are placed into a bath 28 of saline, blood, and/or any other fluid that simulates an in vivo environment. (For example, bath 28 may contain a saline solution having a concentration of NaCl, by weight, of between 0.45% and 1.8%.) Subsequently, while the electrode and the piece of biological tissue are coupled to one another in bath 28, the piece of biological tissue is ablated, using ablation probe 20. In particular, an electric current, which is generated by generator 21, is passed between the ablation probe—specifically, an ablation electrode 32 at the distal end of the probe—and the indifferent electrode.

Using the method depicted in FIG. 1, different ablation probes may be compared to each other during the design process. Thus, for example, after using probe 20 to ablate the biological tissue, parameters such as coagulation and steam pop rates, temperatures measured at the surface and/or interior of the biological tissue, and lesion sizes may be recorded. Subsequently, another probe, having a different design, may be used to ablate another piece of biological tissue, and the same parameters may be recorded and compared to the previously-recorded parameters. Based on this comparison, the superior ablation-probe design may be identified.

The layout of electrode 22 is depicted in FIGS. 2A-B, which are schematic illustrations of cross-sections through indifferent electrodes, in accordance with some embodiments of the present invention.

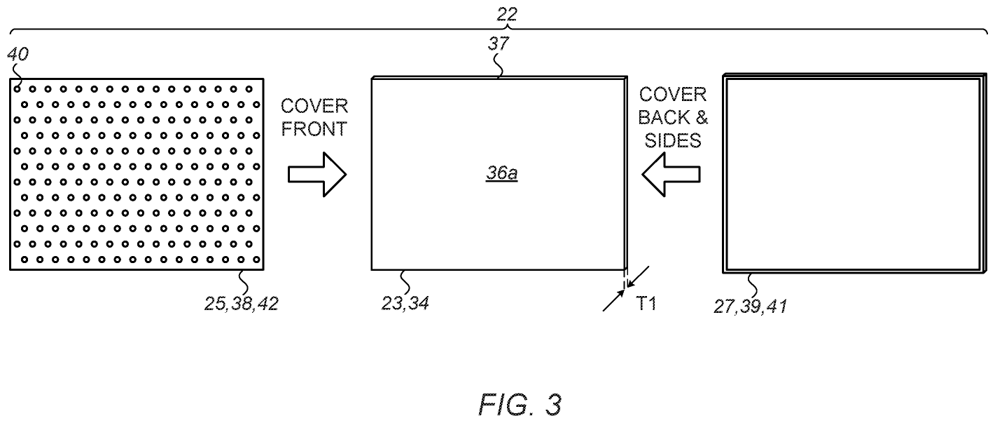

In general, as illustrated in FIGS. 2A-B, indifferent electrode 22 comprises three layers: (i) an electrically-conductive layer 23, comprising a first face 36a and a second face 36b that are opposite one another, (ii) a first electrically-insulative layer 25 that is shaped to define a plurality of apertures 40, and that covers first face 36a without covering portions 31 of the first face that are aligned with the apertures, and (iii) a second electrically-insulative layer 27 that covers second face 36b, typically without exposing any portion of the second face. (As further described below with reference to FIG. 3, second electrically-insulative layer 27 may further cover the sides of electrically-conductive layer 23.)

Prior to utilizing electrode 22, electrically-conductive layer 23 is connected to ground, as described above with reference to FIG. 1. Additionally, the electrode is coupled to the piece of biological tissue such that first face 36a (and first electrically-insulative layer 25) face the tissue, typically with the first electrically-insulative layer 25 contacting the tissue. The electrode may be strapped to the tissue (as shown in FIG. 1), glued to the tissue via an adhesive applied to first electrically-insulative layer 25, and/or coupled to the tissue in any other suitable way. Typically, the electrode is flexible, such that the electrode may conform to the curvature of the tissue.

Typically, the electrode and the piece of biological tissue are similarly sized and shaped. For example, the difference between (i) the total surface area of first face 36a, and (ii) the surface area of the surface of the tissue to which the electrode is coupled, may be less than approximately 25% of the total surface area of first face 36a.

Typically, to help attain a uniform impedance, apertures are densely and uniformly distributed over first electrically-insulative layer 25. For example, the distance between any given aperture and the aperture that is closest to the given aperture may be less than approximately 6 mm, such as less than approximately 4 mm. Nonetheless, the apertures are relatively small, such that the combined surface area of portions 31 of first face 36a is less than approximately 1%, such as less than approximately 0.5%, of the total surface area of the first face. For example, assuming that first face 36a and first electrically-insulative layer 25 each have a total surface area of A0, the combined area of apertures 40 may be less than approximately 0.01*A0, such that less than approximately 1% of first face 36a is aligned with the apertures. Thus, the impedance seen by generator 21 (FIG. 1) may be similar to the impedance that would be seen in vivo.

As a purely illustrative example, if the size of first electrically-insulative layer 25 is 3 cm×3 cm, the first electrically-insulative layer may be shaped to define 49 apertures (e.g., arranged in a 7×7 grid), each aperture having an area of between approximately 0.02 and approximately 0.09 mm², such that between approximately 0.1% and approximately 0.5% of first face 36a is aligned with the apertures. If the size of first electrically-insulative layer 25 is 10 cm×10 cm, the first electrically-insulative layer may be shaped to define 2500 apertures (e.g., arranged in a 50×50 grid), each aperture having an area of between approximately 0.004 and approximately 0.02 mm², such that between approximately 0.1% and approximately 0.5% of first face 36a is aligned with the apertures.

In some embodiments, apertures 40 are arranged in a rectangular grid. In other embodiments, as shown in FIG. 3 (described below), the apertures are arranged in a hexagonal close-packed pattern. (Advantageously, such a pattern may facilitate a larger number of apertures, relative to a grid.) Alternatively, the apertures may be arranged in any other suitable pattern.

In some embodiments, as shown in FIG. 2B, electrode 22 further comprises respective electrically-conductive metallic deposits 33 that contact electrically-conductive layer 23 (particularly, portions 31 of first face 36a) and at least partly fill apertures 40. In some embodiments, metallic deposits 33 comprise the same material(s) as does electrically-conductive layer 23. In other embodiments, metallic deposits 33 comprise a different material. In such embodiments, metallic deposits 33 may help slow or prevent the oxidation of the electrically-conductive layer, in the event that electrically-conductive layer 23 comprises copper and/or another metal that is readily oxidized. For example, metallic deposits 33 may comprise gold and/or any other metal that is generally inert.

In some embodiments, as further shown in FIG. 2B, metallic deposits 33 further cover respective portions of first electrically-insulative layer 25 that surround the apertures. For example, if each aperture is shaped to define a circle, each metallic deposit may cover a larger circular area having a diameter that is up to 500% larger than the diameter of the aperture. The deposition of the metallic deposits on the surface of the first electrically-insulative layer may help reduce the impedance seen by the generator, in the event that the impedance “provided” by apertures 40 is too high.

Each layer in electrode 22 may have any suitable shape, such as a rectangular shape. Typically, the total surface area of first face 36a (which is generally equal to that of second face 36b) is at least 9 cm², such as at least 30 cm², 50 cm², 70 cm², or 90 cm².

In general, each layer of electrode 22 may be made of any suitable material, and the layers may be combined using any suitable manufacturing procedure. Some specific examples are described in the following subsections of the description.

### Using a Covered Electrically-Conductive Plate

Reference is now made to FIG. 3, which is a schematic exploded view of electrode 22, in accordance with some embodiments of the present invention.

In some embodiments, electrically-conductive layer 23 comprises an electrically-conductive plate 34, which may also be referred to as a “substrate” or a “sheet.” Plate 34 may comprise brass, bronze, stainless steel, and/or any other suitable conducting metallic or non-metallic material.

In addition to first face 36a and second face 36b, plate 34 comprises one or more side faces 37, which are disposed between the first face and second face of the plate. (First face 36a, which is shown in FIG. 3, is referred to in FIG. 3 as the “front” of plate 34, while second face 36b, which is not shown, is referred to as the “back” of the plate.) Typically, the thickness T1 of plate 34—i.e., the distance between the first face and the second face of the plate—is less than 0.5 mm, such that side faces 37 have a much smaller surface area than that of the first face or second face of the plate. By virtue of the thinness of the plate, and/or by virtue of being made of a flexible or conformable material (e.g., a flexible conductive polymer sheet), plate 34 may conform to the curvature of the biological tissue to which the plate is coupled.

In these embodiments, first electrically-insulative layer 25 comprises a first electrically-insulative cover 38, which is shaped to define apertures 40. Cover 38 is coupled to first face 36a, such that cover 38 covers the majority of the first face, but does not cover those portion of the first face that are aligned with apertures 40.

In some embodiments, as depicted in FIG. 3, cover 38 comprises a perforated electrically-insulative sheet 42, comprising, for example, a plastic. In such embodiments, apertures 40, which may also be referred to as “perforations,” may be formed by laser-drilling through sheet 42. To couple the sheet to first face 36a, a suitable adhesive may be applied to the inner face of the sheet and/or to the first face of the plate, and the inner face of the sheet may then be stuck to the plate. Alternatively, the sheet may comprise an inner adhesive layer, such that, following the perforation of the sheet, the sheet may stick to the plate without the need to first apply an adhesive. (Typically, sheet 42 is perforated before the sheet is coupled to first face 36a.)

In other embodiments, cover 38 comprises an electrically-insulative coating that coats first face 36a, such as a layer of electrically-insulative paint that is painted onto first face 36a. In such embodiments, apertures 40 may be formed by laser-ablating the coating.

Similarly, second electrically-insulative layer 27 comprises a second electrically-insulative cover 39, which covers the second face of plate 34. Typically, the second cover also covers side faces 37 of the plate. Cover 39 may comprise, for example, one or more strips of dicing tape or polyimide tape, or an electrically-insulative coating, such as a layer of electrically-insulative paint. Alternatively, cover 39 may comprise at least one unperforated electrically-insulative sheet 41. (As shown in FIG. 3, the edges of sheet 41 may be folded, so as to cover side faces 37.) Sheet 41 may comprise an inner adhesive layer that adheres to plate 34; alternatively, sheet 41 may be adhered to plate 34 using an applied adhesive.

In some embodiments, the first and second electrically-insulative covers are continuous with one another. For example, a continuous electrically-insulative coating may be applied over the entire surface of plate 34. Subsequently, apertures 40 may be formed over first face 36a by ablating the coating, as described above. As another example, electrode 22 may comprise an electrically-insulative case, such as a folded sheet of plastic, comprising both a perforated flap and an unperforated flap. Prior to using the electrode, plate 34 may be inserted into the case, and the case may then be sealed shut.

As described above with reference to FIG. 2B, metallic deposits 33 may be deposited into apertures 40 and, optionally, onto the surface of cover 38. For example, following the covering of the plate, the plate may be inserted into a plating bath for a particular duration of time, such that a plating material (e.g., gold) contained in the bath attaches to the exposed portions of the plate, at least partly fills the apertures, and then, optionally, radiates outward from the apertures over the surface of cover 38. Alternatively, any other suitable technique, such as a sputtering technique, may be used to deposit the metallic deposits.

### Using a Coated Electrically-Insulative Substrate

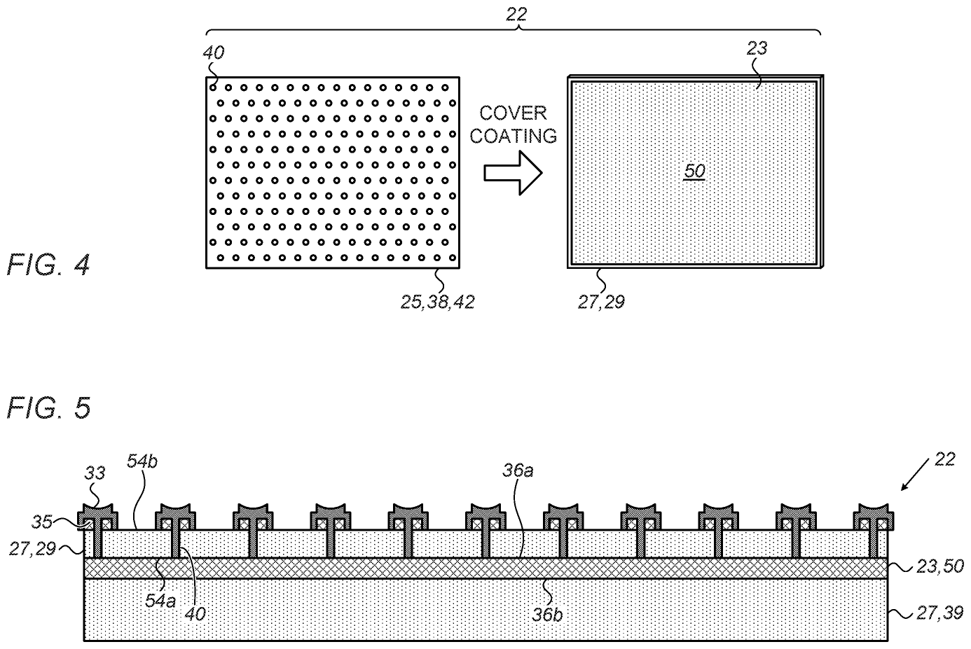

Reference is now made to FIG. 4, which is a schematic exploded view of electrode 22, in accordance with other embodiments of the present invention.

In FIG. 4, second electrically-insulative layer 27 comprises an electrically-insulative substrate 29, comprising, for example, a flexible insulative polymer, such as a polyimide. In such embodiments, electrically-conductive layer 23 comprises an electrically-conductive coating 50 that coats substrate 29. As in the case of FIG. 3, first electrically-insulative layer 25 comprises cover 38 (comprising, for example, sheet 42 or an electrically-insulative coating), which is coupled to electrically-conductive coating 50.

Coating 50 may be sputtered or rolled onto substrate 29. Alternatively, coating 50 may comprise a vapor deposition coating. In some embodiments, coating 50 comprises copper. For example, electrode 22 may comprise a flexible copper-coated polyimide substrate of the type used for flexible printed circuit boards (PCBs).

As described above with reference to FIGS. 2B and 3, metallic deposits 33 may be deposited into apertures 40, e.g., using the plating technique described above.

Reference is now made to FIG. 5, which is a schematic illustration of a cross-section through electrode 22, in accordance with yet other embodiments of the present invention.

In FIG. 5, as in FIG. 4, electrode 22 comprises substrate 29, which is coated by coating 50. However, in FIG. 5, substrate 29 functions as first electrically-insulative layer 25, in that the substrate is shaped to define apertures 40. For example, apertures 40 may be laser-drilled through the substrate. Second electrically-insulative layer 27 comprises cover 39, which is coupled to coating 50.

In some embodiments, both the first surface 54a and the second surface 54b of the substrate, which are opposite one another, are initially coated with an electrically-conductive metal, typically copper. Subsequently, the coating is removed (e.g., etched away) from second surface 54b, except for those portions of second surface 54b that surround the apertures. Electrode 22 thus comprises a plurality of electrically-conducting islands 35 that coat respective portions of second surface 54b that surround the apertures. (The cross-section in FIG. 5 runs through a row of apertures, such that each island appears as two segments positioned at alternate sides of a respective aperture.) For example, if each aperture is circular, each island may be shaped to define a torus that surrounds the aperture.

Next, typically using the above-described plating technique, a metallic substance is deposited into apertures 40, such that electrode 22 comprises respective metallic deposits 33 that fill the apertures and connect coating 50 to islands 35. Typically, as shown in FIG. 5, metallic deposits 33 further cover the islands.

### Using the Electrode In Vivo

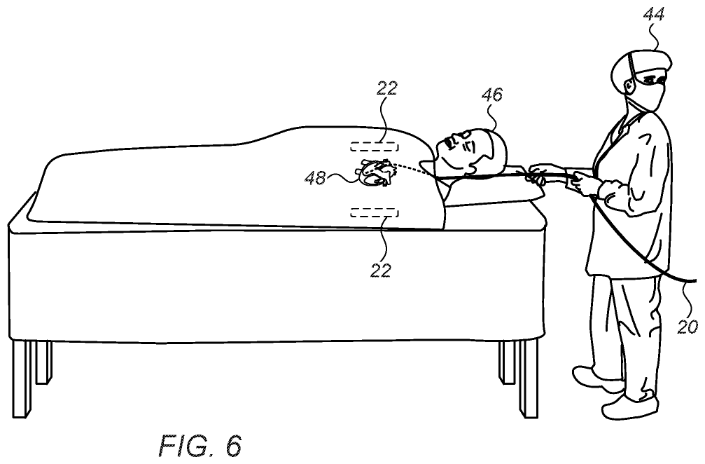

Reference is now made to FIG. 6, which is a schematic illustration of an ablation procedure, in accordance with some embodiments of the present invention. In particular, FIG. 6 depicts a cardiac ablation procedure, in which an operating physician 44 uses ablation probe 20 to ablate cardiac tissue, such as myocardial tissue, of the heart 48 of a subject 46.

As described above in the Overview, in addition to being used in vitro, electrode 22 may be used in vivo. For example, one or more electrodes 22 may function as indifferent electrodes for the cardiac ablation procedure depicted in FIG. 6. First, using any suitable adhesive, and/or any suitable strap(s), the electrodes are coupled to the body of subject 46 such that the first face of the electrically-conducting layer of each of the electrodes faces the subject. (Each of the electrodes is proximally connected to electrical ground, as in FIG. 1.) For example, as depicted in FIG. 6, a first electrode may be coupled to the chest of the subject, and a second electrode may be coupled to the back of the subject. Alternatively, two or more electrodes 22 may be spatially distributed over the body in any other suitable way, such that the impedance seen by the generator does not vary significantly as a function of the position or orientation of the probe within the subject's body. (Typically, as described above with reference to FIGS. 2A-B, the electrodes are flexible, such that the electrodes may conform to the curvature of the subject's body.)

Subsequently to coupling the electrodes to the subject, physician 44 inserts probe 20 into the body of the subject, such that, for example, ablation electrode 32 (FIG. 1) is within heart 48. (As in FIG. 1, probe 20 is proximally connected to signal generator 21.) Next, while electrodes 22 are coupled to the subject, the physician, using probe 20, ablates the tissue of the subject, by passing an electric current between the ablation probe (specifically, the ablation electrode) and the indifferent electrodes.

It is noted that the techniques described hereinabove with reference to the cardiac ablation procedure depicted in FIG. 6 may be similarly applied to other types of ablation procedures. For example, one or more electrodes 22 may function as indifferent electrodes for an otolaryngological or a neurological ablation procedure. To help attain a uniform impedance, the electrodes may be spatially distributed in the vicinity of the otolaryngological or neurological tissue that is to be ablated; for example, one electrode may be coupled to the subject's forehead, and another electrode may be coupled to the nape of the subject's neck.

It will be appreciated by persons skilled in the art that the present invention is not limited to what has been particularly shown and described hereinabove. Rather, the scope of embodiments of the present invention includes both combinations and subcombinations of the various features described hereinabove, as well as variations and modifications thereof that are not in the prior art, which would occur to persons skilled in the art upon reading the foregoing description. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. Apparatus, comprising:
an electrically-conductive layer, comprising a first face and a second face that are opposite one another;
a first electrically-insulative layer that is shaped to define a plurality of apertures and that covers the first face without covering portions of the first face that are aligned with the apertures; and
a second electrically-insulative layer that covers the second face.

2. The apparatus according to claim 1,
wherein the electrically-conductive layer comprises an electrically-conductive plate,
wherein the first electrically-insulative layer comprises a first electrically-insulative cover coupled to the first face of the plate, and
wherein the second electrically-insulative layer comprises a second electrically-insulative cover coupled to the second face of the plate.

3. The apparatus according to claim 2, wherein the plate comprises one or more side faces disposed between the first face and the second face, and wherein the second electrically-insulative cover covers the side faces.

4. The apparatus according to claim 2, wherein the apparatus comprises an electrically-insulative case that comprises the first cover and the second cover.

5. The apparatus according to claim 1,
wherein the second electrically-insulative layer comprises an electrically-insulative substrate,
wherein the electrically-conductive layer comprises an electrically-conductive coating that coats the electrically-insulative substrate, and
wherein the first electrically-insulative layer comprises an electrically-insulative cover coupled to the electrically-conductive coating.

6. The apparatus according to claim 5, wherein the electrically-conductive coating comprises a vapor deposition coating.

7. The apparatus according to claim 5, wherein the electrically-insulative substrate comprises a polyimide.

8. The apparatus according to claim 7, wherein the electrically-conductive coating comprises copper.

9. The apparatus according to claim 1,
wherein the first electrically-insulative layer comprises an electrically-insulative substrate,
wherein the electrically-conductive layer comprises an electrically-conductive coating that coats the electrically-insulative substrate, and
wherein the second electrically-insulative layer comprises an electrically-insulative cover coupled to the electrically-conductive coating.

10. The apparatus according to claim 9, wherein the electrically-insulative substrate comprises a polyimide.

11. The apparatus according to claim 9, wherein the electrically-conductive coating comprises copper.

12. The apparatus according to claim 9,
wherein the electrically-insulative substrate comprises a first surface and a second surface that are opposite one another,
wherein the electrically-conductive coating coats the first surface of the electrically-insulative substrate, and
wherein the apparatus further comprises:
a plurality of electrically-conducting islands that coat respective portions of the second surface of the electrically-insulative substrate that surround the apertures; and
respective metallic deposits that fill the apertures and electrically connect the electrically-conductive coating to the islands.

13. The apparatus according to claim 12, wherein the metallic deposits further cover the islands.

14. The apparatus according to claim 1, further comprising respective electrically-conductive metallic deposits that contact the electrically-conductive layer and at least partly fill the apertures.

15. The apparatus according to claim 14, wherein the metallic deposits comprise gold.

16. The apparatus according to claim 14, wherein the metallic deposits further cover respective portions of the first electrically-insulative layer that surround the apertures.

17. The apparatus according to claim 1, wherein a combined surface area of the portions of the first face that are aligned with the apertures is less than approximately 1% of a total surface area of the first face.

18. The apparatus according to claim 17, wherein the combined surface area of the portions of the first face that are aligned with the apertures is less than approximately 0.5% of the total surface area of the first face.

19. The apparatus according to claim 17, wherein a distance between any one of the apertures and another, closest one of the apertures is less than approximately 6 mm.

20. The apparatus according to claim 17, wherein the total surface area of the first face is at least 9 cm².

21. The apparatus according to claim 1, wherein the apertures are arranged in a rectangular grid.

22. The apparatus according to claim 1, wherein the apertures are arranged in a hexagonal close-packed pattern.

23. The apparatus according to claim 1, wherein the electrically-insulative cover comprises a perforated electrically-insulative sheet.

24. The apparatus according to claim 1, wherein the electrically-insulative cover comprises an electrically-insulative coating.

25. The apparatus according to claim 24, wherein the electrically-insulative coating comprises a layer of electrically-insulative paint.

26. A method for testing an ablation probe, the method comprising:
providing an electrode that includes:
an electrically-conductive layer, including a first face and a second face that are opposite one another,
an electrically-insulative cover that is shaped to define a plurality of apertures and that covers the first face without covering portions of the first face that are aligned with the apertures, and
an electrically-insulative layer that covers the second face;
coupling the electrode and a piece of biological tissue to one another such that the first face faces the piece of biological tissue;
placing the electrode and the piece of biological tissue into a bath; and
while the electrode and the piece of biological tissue are coupled to one another in the bath, using the ablation probe, ablating the piece of biological tissue by passing an electric current between the ablation probe and the electrode.

27. The method according to claim 26, wherein the first face faces a surface of the piece of biological tissue, and wherein a difference between (i) a total surface area of the first face, and (ii) a surface area of the surface of the piece of biological tissue, is less than approximately 25% of the total surface area of the first face.

28. A method, comprising:
providing one or more electrodes, each of the electrodes including:
an electrically-conductive layer, including a first face and a second face that are opposite one another,
an electrically-insulative cover that is shaped to define a plurality of apertures and that covers the first face without covering portions of the first face that are aligned with the apertures, and
an electrically-insulative layer that covers the second face;
coupling each of the electrodes to a body of a subject such that the first face faces the subject; and
while the electrodes are coupled to the body of the subject, using an ablation probe disposed within the body, ablating tissue of the subject by passing an electric current between the ablation probe and the electrodes.

29. The method according to claim 28, wherein coupling each of the electrodes to the body of the subject comprises coupling a first one of the electrodes to a chest of the subject and a second one of the electrodes to a back of the subject.

30. The method according to claim 28, wherein coupling each of the electrodes to the body of the subject comprises coupling a first one of the electrodes to a forehead of the subject and a second one of the electrodes to a nape of a neck of the subject.

31. The method according to claim 28, wherein the tissue is of a type selected from the group of tissue types consisting of: cardiac tissue, otolaryngological tissue, and neurological tissue.
